(19) United States
(12) Patent Application Publication
(10) Pub. No.: US 2006/0211721 A1
(43) Pub. Date: Sep. 21, 2006

(54) Nutraceutical formulation of a cognitive enhancement system

(76) Inventor: Alan R. Roberts, Los Angeles, CA (US)

Correspondence Address: ARENT FOX PLLC, 1050 CONNECTICUT AVENUE, N.W., SUITE 400, WASHINGTON, DC, 20036, US

(21) Appl. No.: 11/084,189

(22) Filed: Mar. 21, 2005

(51) Int. Cl. A61K 31/51; A61K 31/473; A61K 31/455; A61K 31/198

(52) U.S. Cl. 514/276; 514/295; 514/356; 514/561

(57) ABSTRACT

A nutraceutical composition and method of use thereof includes core ingredients, huperzine A, dimethylaminoethanol (DMAE), thiamin, niacin, and pantothenic acid. The core ingredients provide a synergistic effect to improve and support cognitive function, such as learning, thinking, and judging and to promote alertness, awareness, attention and mental clarity. Combinations of additional ingredients, such as vinpocetine, acetyl-L-carnitine, L-pyroglutamic acid, pyridoxine, folic acid, vitamin B₁₂, phosphatidylserine, L-theanine, and rhodiola rosea can be added to the core ingredients to enhance the beneficial and synergistic effects of the composition.

### FIELD OF THE INVENTION

The present invention relates to a nutraceutical formulation with cognitive support compounds that promote enhanced cognitive performance.

### BACKGROUND OF THE INVENTION

Attempts to enhance cognitive performance in individuals have a long history. However, nutraceutical interventions have had limited success. A substantial amount of research in the area of cognitive enhancement has been conducted to determine the efficacy of natural nutraceutical agents (commonly referred to as “nutraceuticals” or “dietary supplements”), such as Ginkgo biloba and ginseng, among others.

The FDA has defined a dietary supplement to be a product (other than tobacco) that is intended to supplement the diet that bears or contains one or more of the following dietary ingredients: a vitamin, a mineral, an herb or other botanical, an amino acid, a dietary substance for use by man to supplement the diet by increasing the total daily intake, or a concentrate, metabolite, constituent, extract, or combinations of these ingredients. See, FDA Center for Food Safety and Applied Nutrition. Dietary Supplement Health Education Act of 1994. Dorland's Illustrated Medical Dictionary (30^{th} ed.) defines nutrient as “a food or other substance that provides energy or building material for the survival and growth of a living organism.” Accordingly, as used herein, a “nutraceutical” is any substance that provides physiological, medical or health benefits or protection.

The research into the efficacy of nutraceuticals has investigated the degree to which nutraceuticals may have practical application in short- and long-term cognitive enhancement or cognitive activity (herein collectively referred to as “cognitive function”). The research that has been conducted in this area has revealed some beneficial qualities in certain substances. For example, through this research, nutraceuticals, such as Ginkgo biloba and Bacopa monnieri have been shown to improve cognitive function to some degree.

Other substances that have been studied in the same context are discussed below.

Huperzine A (generally referred to herein as “huperzine”), in particular, is an alkaloid found in the herb Huperzia serrata and is generally believed to improve cognitive function. While not wishing to be bound by this theory, the nutraceutical effect of huperzine may be linked to the presence and activity of acetylcholine, which is known to be a neurotransmitter in the central and peripheral nervous system that appears to be correlated to cognition and memory. In particular, the loss of acetylcholine function appears to be a significant factor in several disorders of brain function, including Alzheimer's disease, which is believed to be a condition where there is a relative shortage of acetylcholine. Researchers believe that huperzine is an acetylcholinesterase (AChE) inhibitor, blocking the breakdown of acetylcholine. By inhibiting the enzyme that breaks down acetylcholine, more acetylcholine becomes available to stimulate neurons.

Huperzine is known to have additional beneficial properties. For instance, huperzine is known to be a reversible butyrylcholinesterase inhibitor. Also, huperzine has demonstrated good penetration through the blood brain barrier, high oral bioavailability, and long durations of inhibitory action on acetylcholinesterase. Furthermore, huperzine is also believed to protect neurons from cell death caused by toxic levels of glutamate.

Another substance thought to improve cognitive function is 2-dimethylaminoethanol (DMAE), which is a chemical known to be produced in the brain. Researchers have linked the nutraceutical properties of DMAE to the presence and activity of choline. Specifically, researchers have noted that DMAE is structurally similar to choline, which is a precursor to acetylcholine, and which is believed to be important to cognitive function, as previously described. It is believed that DMAE stimulates the production of choline. It is also believed that DMAE is correlated to slowing cell membrane degradation.

Thiamin (also known as vitamin B₁) is known to be critical in the conversion of blood sugar, i.e., glucose, into cellular energy and it has been implicated in neurotransmission and nerve conduction. The metabolic activities of thiamin primarily affect the nerves, muscles and cardiovascular system. It is also known that thiamin is involved in the synthesis of acetylcholine.

Niacin (also known as vitamin B₃) functions metabolically as a component of two important coenzymes: nicotinamide adenine dinucleotide (NAD) and nicotinamide adenine dinucleotide phosphate (NADP), which play an essential role in over 200 chemical reactions in the body, including oxidation-reduction reactions, ATP synthetic pathways, and ADP-ribose transfer reactions. Niacin has been linked to enhancing brain choline concentrations.

Moreover, pantothenic acid (also known as vitamin B₅) is generally converted to a sulfur-containing compound called pantetheine, which is further converted to coenzyme A. Coenzyme A is linked to cellular metabolism through energy-yielding oxidation of glycolytic products and other metabolites through the mitochondrial tricarboxylic acid cycle. Pantothenic acid is also critical to the synthesis of essential molecules, such as fatty acids, membrane phospholipids, and some amino acids.

Other compounds have also been studied, and have been correlated with the activity of brain and spinal tissues and possibly cognitive function. For example, L-pyroglutamic acid (PCA or PGA) is chemically related to pyroglutamate, which is an amino acid normally present in large amounts in human brain, cerebral spinal fluid and blood. Additionally, acetyl-L-carnitine (ALC) is known to facilitate the production of energy from long chain fatty acids, and acetyl-L-carnitine is considered to be a cognitive enhancing nutrient because it increases the production and release of acetylcholine in the brain. Some research has shown that acetyl-L-carnitine acts as a neuroprotective agent, including reversing hippocampal and prefrontal neuronal loss and lipofuscin accumulation in animals and slowing the decline in the production of nerve growth factor (NGF) and NGF receptors. Moreover, vinpocetine is believed to be a potent vasodilator and has been commonly used in the treatment of cerebrovascular disorders.

Nutraceutical compositions, which have been believed to enhance cognitive function, have been produced and commercialized for many years. Many of these conventional nutraceutical compositions are known to use numerous vitamins, compounds, and herbs to provide cognitive benefits to the user. These compositions are colloquially referred to as “cocktails” because these compositions combine an array of active ingredients in seemingly arbitrary dosages. Also, because of the arbitrary nature of the compositions, the potential for synergistic effects between the ingredients is unpredictable. For example, typically these compositions combine ingredients, such as vitamins, minerals, and natural herbs, each of which is believed in folklore or by conventional wisdom to support cognitive function. However, as is often the case, the ingredients are unproven in terms of actual effectiveness, and sometimes safety.

The present state of the art includes nutraceutical compositions that contain DMAE, which is typically in the form of DMAE bitartrate, huperzine, thiamin, niacin, or pantothenic acid, among other ingredients. However, notably, these compositions contain low amounts of DMAE and variable amounts of huperzine. Moreover, these compounds have not been shown to have significant effects on cognitive function.

Accordingly, there is an unmet need in the art to create a nutraceutically effective composition to promote cognitive function. In particular, there is an unmet need in the art for a composition having ingredients that enhance cognitive function or generally increase alertness and awareness. Additionally, there is an unmet need in the art to create a nutraceutically effective composition with other clinically-tested ingredients, such as acetyl-L-carnitine, L-pyroglutamic acid, and vinpocetine. There is also an unmet need in the art to create compositions with core ingredients and additional ingredients that are clinically proven to enhance cognitive function, be synergistically effective, and be safe.

### SUMMARY OF THE INVENTION

The present invention overcomes the above-mentioned problems by providing a cholinergic and synergistic nutraceutical composition having increased effectiveness as compared to systems currently available.

A first embodiment of the present invention provides a composition to improve cognitive function in humans comprising an effective amount of huperzine, DMAE, thiamin, niacin and pantothenic acid or nutraceutically acceptable derivatives thereof. As used herein, “nutraceutically acceptable” means generally recognized as safe and not interfering or otherwise affecting the medical and health benefits of the nutraceutical as described herein. The composition optionally further comprises one or more nutraceutically acceptable carriers or excipients, as needed. The composition of this embodiment is useful for improving various brain functions, and/or generally supporting cognitive function.

The composition exhibits a synergistic effect not known in the present art when delivered in the prescribed ranges. In a variation, the nutraceutically acceptable forms of huperzine, DMAE, thiamin, niacin and pantothenic acid are combined to produce a synergistic effect promoting cognitive function. In additional variations, nutraceutically acceptable ingredients are added to the formulation to produce improved synergistic cognitive effects and cholinergic effects.

In one variation of the present invention, DMAE is preferably administered as DMAE bitartrate at a dose greater than 100 to about 1500 mg/day of DMAE bitartrate. Huperzine is administered at a dose from about 0.01 to about 1 mg/day. Thiamin is administered in the amount from about 25 to about 125 mg/day. Niacin is administered in the amount from about 100 to about 300 mg/day. Pantothenic acid is administered in an amount greater than 100 to about 300 mg/day.

In another variation, the composition is combined with nutraceutically acceptable carriers or excipients to assist oral, dermal, or parenteral delivery. In one more variation, the composition is delivered orally in a tablet and/or capsule. In yet another variation, the dosages of the delivery system for the compositions may be broken down into two to six administrations (e.g., doses) per day, wherein the doses in each administration are proportional to the total daily dosage.

In a second embodiment of the present invention, the effective amounts of the core ingredients of huperzine, DMAE, thiamin, niacin, and pantothenic acid, or nutraceutically acceptable derivatives thereof, are combined with nutraceutically effective amounts of vinpocetine, acetyl-L-carnitine, and/or pyroglutamic acid. In variations of this embodiment, huperzine, DMAE, thiamin, niacin, and pantothenic acid are combined with only vinpocetine, only acetyl-L-carnitine, only pyroglutamic acid, only vinpocetine and pyroglutamic acid, only acetyl-L-carnitine and pyroglutamic acid, or only vinpocetine and acetyl-L-carnitine.

This embodiment of the present invention exhibits a synergistic effect not presently known in the art when delivered in the prescribed ranges. If present, vinpocetine is preferably administered in an amount from about 10 to about 30 mg/day. If present, acetyl-L-carnitine is preferably administered in the form of acetyl-L-carnitine hydrochloride (HCL) in an amount from about 500 to about 2000 mg/day acetyl-L-carnitine hydrochloride (HCL). If present, pyroglutamic acid is administered in an amount from about 250 to about 750 mg/day.

A third embodiment combines the variations and permutations of the previous embodiments with rhodiola rosea root (generally referred to herein as “rhodiola rosea”) or a nutraceutically acceptable composition thereof. If present, rhodiola rosea is preferably administered in an amount from about 200 to about 600 mg/day. The mode of administration and dosage are consistent with the previously disclosed embodiments.

Other embodiments of the present invention incorporate the previously presented embodiments with combinations of effective amounts of pyridoxine (vitamin B₆), folic acid, vitamin B₁₂, and phosphatidylserine (PS), or their nutraceutically accepted derivatives or salts.

If present, pyridoxine (B₆) is preferably administered in an amount from about 10 to about 50 mg/day. If present, folic acid is preferably administered in an amount from about 0.4 to about 2 mg/day. If present, vitamin B₁₂ is preferably administered in an amount from about 0.5 to about 1.5 mg/day. If present, phosphatidylserine is preferably administered in an amount from about 50 to about 300 mg/day. The mode of administration and dosage are consistent with the previously disclosed embodiments.

In yet other embodiments, L-theanine is combined with the core formulation of huperzine, DMAE, thiamin, niacin, and pantothenic acid and/or other embodiments including the above-identified nutraceuticals. If present, L-theanine is preferably administered in an amount from about 50 to about 250 mg/day. The mode of administration and dosage are consistent with the previously disclosed embodiments.

Still other embodiments of the present invention include a method of improving cognitive function in a subject, comprising administering to the subject a composition having an effective amount of huperzine, DMAE, thiamin, niacin and pantothenic acid or their nutraceutically acceptable forms, and optionally, nutraceutically acceptable excipients. In variations, a method of improving cognitive function of a subject includes administering an effective dose of huperzine, DMAE, thiamin, niacin, and pantothenic acid, in combination with an effective dose of one or more nutraceuticals selected from vitamin B₆, folic acid, vitamin B₁₂, phosphatidylserine, L-theanine, rhodiola rosea, vinpocetine, acetyl-L-carnitine, and/or pyroglutamic acid.

Any of the embodiments illustrated above and below stand independently or features may be combined to achieve preferred embodiments. Additional advantages and novel features of the invention will also become more apparent to those skilled in the art upon examination of the following or upon learning by practice of the invention.

### DESCRIPTION OF THE PREFERRED EMBODIMENTS

The present invention overcomes the above-mentioned problems, as well as others, by providing a more nutraceutically and synergistic system as compared to systems currently available.

The present invention overcomes the above-mentioned problems, as well as others, by providing a synergistically effective composition comprising DMAE, huperzine, thiamin, niacin and pantothenic acid (also collectively referred to herein as the “core ingredients” or “core formulation”) to improve and support cognitive function. The present invention, as presented in the core formulation and other formulations in the amounts discussed herein, is useful for improving or supporting various cognitive functions, such as learning, thinking, and judging, as well as promoting focus, attention, alertness, awareness, and mental clarity. It is believed that the composition promotes cholinergic properties in the brain and supports acetylcholine levels by (1) enhancing the brain level of choline, an acetylcholine precursor; (2) contributing to the structure of acetylcholine; (3) increasing the production and release of acetylcholine; and, (4) inhibiting acetylcholinesterase activity.

In embodiments of the present invention, a formulation comprises an effective amount of the core ingredients and optionally one or more nutraceutically acceptable carriers or excipients. The core ingredients are preferably refined for nutraceutical use.

Each core ingredient may be comprised of or formed from acceptable forms, including salts or derivatives thereof. Refined and nutraceutically acceptable forms of DMAE, huperzine, thiamin, niacin and pantothenic acid can be prepared by a method known in the art. For example, thiamin may be implemented as thiamin HCl or a salt thereof. Additionally, for example, pantothenic acid may be implemented as calcium pantothenate. In one variation, two or more core ingredients are blended.

According to one variation of the present invention, DMAE (otherwise referred to as DMAE) is preferably administered at a dose greater than 38 to about 500 mg/day, preferably from about 60 to about 150 mg/day, and more preferably from about 75 to about 120 mg/day. In an even more preferred embodiment, DMAE, in conjunction with at least the core ingredients, is administered in a dose of about 100 mg/day. In the most preferred embodiment, DMAE, in conjunction with at least the core ingredients, is administered in a dose of about 111 mg/day.

Preferably, DMAE is administered in the form of DMAE bitartrate, which contains about 37% DMAE. Accordingly, when DMAE is administered as DMAE bitartrate, DMAE bitartrate is preferably administered at a dose greater than 100 to about 1500 mg/day, more preferably from about 200 to about 500 mg/day, and even more preferably from about 250 to about 400 mg/day. In the most preferred embodiment, DMAE bitartrate, in conjunction with at least the core ingredients, is administered in a dose of about 300 mg/day.

Other nutraceutically acceptable forms of DMAE now known or later developed may be substituted for the DMAE bitartrate. It is expected that the amount of DMAE in other nutraceutically acceptable forms may vary, as exhibited in the DMAE bitartrate. Nonetheless, the amount of DMAE should be maintained in the nutraceutical formulation.

In accordance with one embodiment of the present invention, huperzine, in combination with at least the remaining core ingredients, is administered at a daily dose from about 0.01 to about 1 mg/day, preferably from about 0.025 to about 0.75 mg/day, more preferably from about 0.05 to about 0.6 mg/day, and more preferably from about 0.075 to about 0.3 mg/day. Even more preferably, huperzine is in the amount of 0.10 to 0.2 mg/day. A most preferred embodiment of the present invention includes about 0.15 mg/day of huperzine.

The vitamins comprising the core composition, thiamin, niacin and pantothenic acid, are generally found at levels greater than the recommended daily allowance (RDA). However, these levels are within a safe range of use. In one variation, the levels of these vitamins are at least 1000% of the RDA.

Specifically, thiamin is preferably administered in the core formulation, as well as in other embodiments, in the amount from about 25 to about 125 mg/day and more preferably from about 50 to about 110 mg/day. More preferably thiamin is in the amount of about 100 mg/day. Niacin is preferably administered, in combination with the other core ingredients (and other potential excipients), in the amount from about 100 to about 300 mg/day and more preferably from about 150 to about 275 mg/day. More preferably niacin is administered in the amount of about 250 mg/day. Pantothenic acid is preferably administered in the amount of greater than 100 to about 300 mg/day and preferably from about 150 to about 275 mg/day. Preferably pantothenic acid is in the amount of 250 mg/day.

With respect to niacin, two forms generally exist: nicotinic acid and niacinamide. Generally, there is a low level of tolerance associated with high-dose (i.e., over 100% of the Daily Value) nicotinic acid. Nicotinic acid may lead to flushing in high doses. Therefore, it is preferable that no more than 35 mg/day or 175% of the Daily Value of nicotinic acid be included in the composition. Alternatively, high-dose niacinamide is generally well tolerated by healthy adults and has been used in retail formulations, as well as, in the clinical study (discussed infra). Preferred embodiments of the present invention use niacinamide. For the purposes of the present invention, nutraceutically acceptable forms of niacin preferably include forms of niacin that are safe at high doses in the amounts presented, i.e., from about 100 to about 300 mg. When forms of niacin that are not recommended at such high amounts, such as nicotinic acid, are used, those forms are preferably present in an amount no greater than 35 mg/day, preferably from about 10 to about 30 mg/day, and more preferably from about 20 to about 25 mg/day.

The “nutraceutically acceptable carrier or excipient” is any carrier or excipient known in the art of nutraceuticals or dietary supplements. Examples of the carrier or excipient include water, aqueous buffer, ethanol, flavoring agents, and physiologically acceptable dyes, as well as carriers suitable for tablets or capsules such as talc, corn starch, rice powder, silica, magnesium stearate, gelatin, titanium dioxide, dextrose, stearic acid, microcrystalline cellulose, crosscarmellose sodium, dicalcium phosphate, and carboxymethyl cellulose. This list is not limiting, however. It would be understood to one skilled in the art to substitute and replace excipients as necessary and/or desired.

In the method of the invention, the composition can be administered orally or parenterally (e.g. dermally, intramuscularly or intravenously). Preferably, the composition is administered orally, e.g., in a liquid, chewing gum, bar, tablet, capsule, powder, or in another chewable form.

The composition of the present invention is preferably in the form of a tablet or capsule having an outer coating to ease oral consumption. The compositions of the tablet and/or capsule, and coatings thereof, are generally known in the art. For example, in one variation, the tablet and coating are comprised of magnesium stearate, croscarmellose sodium, microcrystalline cellulose, stearic acid, silicon dioxide, methylcellulose and glycerin. In another variation, the capsule is comprised of gelatin, rice powder, magnesium stearate and silicon dioxide. Another variation includes any or all of the following or like acting excipients: magnesium stearate, dibasic calcium phosphate, cellulose, stearic acid, silica, hydroxypropylcellulose, cellulose gum, methylcellulose, glycerin, and maltodextrin. Other chemicals, such as silica and rice powder are added as necessary, the addition of which is known in the art. In one variation of the present invention, traces of calcium are included in the composition. Calcium and other like minerals are generally used for processing the tablets and are not considered a functional ingredient of any of the formulations.

The dosages of active ingredients presented above are per day. The delivery of the dosages may be broken down into two to six or more administrations, preferably three to four administrations per day. Accordingly, the delivery mechanism, e.g., the tablet, contains a proportional amount of active ingredients for each administration. A preferred embodiment of the present invention is a tablet with one-third the total dosage taken three times per day. Another preferred embodiment of the present invention is a tablet with one-fourth the total daily dosage taken four times daily.

The core formulation of DMAE, huperzine, thiamin, niacin and pantothenic acid, when used in specific dosages, has a synergistic effect on an individual's overall cognitive function. The dosages of each compound have been chosen to achieve maximal therapeutic efficacy in the individual or multiple levels of cholinergic support. The dosages are at levels discovered to perform as compounds helping cognitive functions, such as paying attention, decision-making, and mental flexibility.

For instance, collectively the core ingredients of DMAE, huperzine, thiamin, niacin and pantothenic acid are believed to have accumulative and synergistic effects related to alertness, concentration, improved mental focus without tiredness, mental endurance improvement, mental adaptation to stressors, enhancement of work performance, learning improvement, and memory improvement. The core ingredients (DMAE, huperzine, thiamin, niacin and pantothenic acid) also allow treated subjects to better adapt to their environmental (internal & external) factors/stressors and to avoid damage from extrinsic and intrinsic factors.

Without being bound by any theory of mechanism of action, the synergism may be caused by a potentiation of the biological effects of DMAE, huperzine, thiamin, niacin and pantothenic acid as related to the permeability of the blood brain barrier and the promotion of acetylcholine.

The combined effect of DMAE, huperzine, thiamin, niacin and pantothenic acid is believed to be greater than the sum of the individual effects of DMAE, huperzine, thiamin, niacin and pantothenic acid administered alone. As a result, in accordance with the invention, DMAE, huperzine, thiamin, niacin and pantothenic acid can be administered at a combined dose lower than what is required of each active ingredient individually, in order to obtain the same levels of beneficial effects. In one variation of the present invention, the core ingredients are provided at the amounts presented above to provide the optimal synergistic effect. In another variation, the amounts of the core ingredients, as presented, provide an elevated synergistic effect while also providing economic value (assessing the relative costs of each ingredient).

Additional embodiments of the formulation of the present invention optionally further comprise supplemental active ingredients. In one such embodiment of the present invention, the core ingredients (huperzine, DMAE, thiamin, niacin, pantothenic acid) are further combined with nutraceutically effective amounts of one or more of the following active ingredients: vinpocetine, acetyl-L-carnitine, and pyroglutamic acid.

Accordingly, in variations of these embodiments, the core ingredients are combined 1) with only vinpocetine; 2) only acetyl-L-carnitine; 3) only pyroglutamic acid. In additional variations, the core ingredients are combined with two of these substances, including: 1) with vinpocetine and pyroglutamic acid; 2) with acetyl-L-carnitine and pyroglutamic acid; and 3) with vinpocetine and acetyl-L-carnitine. In other embodiments of the present invention, the formulation includes the core ingredients combined with vinpocetine, pyroglutamic acid, and acetyl-L-carnitine. Additional nutraceuticals and/or nutraceutically acceptable excipients known in the art may be added to the above combinations.

Each supplemental ingredient may be comprised of or formed from acceptable salts or derivatives thereof. Refined and nutraceutically acceptable forms of vinpocetine, acetyl-L-carnitine, and pyroglutamic acid may be used.

The market generally contains different forms of acetyl-L-carnitine. For example, nutraceutically acceptable forms of acetyl-L-carnitine include acetyl-L-carnitine hydrochloride, acetyl-L-carnitine hydrogen fumarate, acetyl-L-carnitine taurinate hydrochloride, L-ornithine acetyl-L-carnitine dihydrochloride, acetyl-L-carnitine arginate dihydrochloride, and L-leucine acetyl-L-carnitine hydrochloride. Other nutraceutically acceptable forms of acetyl-L-carnitine may also be available or will be available, and should be known to those skilled in the art to be substitutes for acetyl-L-carnitine and the nutraceutically acceptable forms enumerated herein.

In some embodiments, the formulations include an effective amount of core ingredients in combination with effective amounts of supplemental active ingredients: vinpocetine, acetyl-L-carnitine, and pyroglutamic acid. In one variation, vinpocetine is present in the amount from about 10 to about 30 mg/day and preferably from about 15 to about 25 mg/day. More preferably, vinpocetine is present in the amount of 20 mg/day.

In other variations, acetyl-L-carnitine is present in the amount from about 400 to about 1600 mg/day and preferably from about 600 to about 1200 mg/day. More preferably, acetyl-L-carnitine is present in the amount of 800 mg/day.

In more preferred variations, acetyl-L-carnitine is present in the form of acetyl-L-carnitine hydrochloride.

The amount of acetyl-L-carnitine should remain consistent. The amount of acetyl-L-carnitine hydrochloride can be accordingly adjusted by this percentage to determine the corresponding amount of acetyl-L-carnitine. Accordingly, acetyl-L-carnitine hydrochloride can be present in the amount from about 500 to about 2000 mg/day and preferably from about 750 to about 1500 mg/day. More preferably, acetyl-L-carnitine hydrochloride is present in the amount of 1000 mg/day. Generally, the nutraceutically acceptable forms of acetyl-L-carnitine used in some embodiments of this invention can have a potency of about 40% to about 85% of acetyl-L-carnitine.

In yet other variations, pyroglutamic acid is present in the amount from about 250 to about 750 mg/day and preferably from about 350 to about 650 mg/day. More preferably, pyroglutamic acid is present in the amount of about 500 mg/day.

It should be noted that the modes of delivery for these formulations include at least the forms as described above. The frequency with which these formulations are administered is consistent with the description of frequency presented in embodiments above.

In further variations of the present invention, rhodiola rosea is combined with the core ingredients, with each variation including vinpocetine, acetyl-L-carnitine, and L-pyroglutamic acid, and other embodiments described herein. An effective dose of rhodiola rosea provides an adaptigen effect and reduces fatigue. Moreover, rhodiola rosea in combination with cholinergic compounds, such as the core ingredients, is believed to increase cognitive functions, such as decision-making, and to reduce stress.

Rhodiola rosea may optionally be comprised of or formed from acceptable forms or derivatives thereof. Refined rhodiola rosea can be prepared by known methods. In variations of the present invention, rhodiola rosea is in the form of rhodiola rosea extract. In other variations of the present invention, the rhodiola is prepared into a standardized extract including 3% rosavins and 0.8-1.0% salidroside.

In variations including rhodiola rosea extract, rhodiola rosea extract is present in the amount from about 200 to about 600 mg/day and preferably from about 250 to about 450 mg/day. More preferably rhodiola rosea extract is present the amount of 300-400 mg/day.

Moreover, in more embodiments of the present invention, an effective amount of one or more additional active ingredients of the following are combined with the core ingredients and/or the formulations including the supplemental active ingredients, as presented above. Additional active ingredients including pyridoxine (also known as vitamin B₆), folic acid, vitamin B₁₂, and/or phosphatidylserine (PS) are combined with each of the variations presented above.

In variations including these ingredients, the effective amount ranges as follows. Pyridoxine (B₆) is present in the amount from about 10 to about 50 mg/day and preferably from about 20 to about 40 mg/day. More preferably pyridoxine is present in the amount of 30 mg/day. Vitamin B₁₂ is present in the amount from about 0.5 to about 1.5 mg/day and preferably from about 0.6 to about 1.2 mg/day. More preferably vitamin B₁₂ is present in the amount of 1.0 mg/day. Folic acid is present in the amount from about 0.4 to about 2 mg/day. Folic acid is preferably present in the amount greater than 0.4 to about 2 mg/day and more preferably from about 0.8 to about 1.6 mg/day. Even more preferably folic acid is present in the amount of 1.2 mg/day. Phosphatidylserine is present in the amount from about 50 to about 300 mg/day and preferably from about 50 to about 200 mg/day. More preferably phosphatidylserine is in the amount of about 50 mg/day.

In some variations, these additional ingredients (pyridoxine, vitamin B₁₂, folic acid, and PS) may be comprised of or formed from acceptable salts or derivatives thereof. Moreover, these additional ingredients can be refined as known in the art.

The B vitamins used in variations of the present invention were included because they are multiple acetylcholine synthesis co-factors. Accordingly, the presence of these vitamins is believed to increase acetylcholine synthesis. It is further understood that each of these vitamins individually is known to positively affect neural function and that the combination thereof produces a direct or indirect synergistic neural effect.

In yet other embodiment, L-theanine is combined with each of the above-identified variations of the present invention. L-theanine has shown to reduce anxiety and promote dopamine levels. When combined with the core ingredients, for example, L-theanine is believed to increase calmness, attention, and focus.

In still other variations, L-theanine is present in the amount from about 50 to about 250 mg/day and preferably from about 75 to about 200 mg/day. More preferably L-theanine is present in the amount of 100 mg/day.

In additional variations, L-theanine may be comprised of or formed from acceptable salts or derivatives thereof. Refined L-theanine can be prepared by a method known in the art.

It should be noted that the modes of delivery for these formulations includes at least the forms as described above. The frequency with which these formulations are administered is consistent with the description of frequency presented above.

Other embodiments of the invention are directed to methods for (a) increasing brain speed; (b) increasing alertness; (c) improving mental focus and attention; and (d) enhancing decision-making, by administering effective amounts, preferably synergistic effective amounts of the core formulation (DMAE, huperzine, thiamin, niacin and pantothenic acid) to a subject in need thereof. Additional embodiments include treating subjects with effective amounts of embodiments presented above to improve cognitive function.

The method can be practiced by administering a dosage of one embodiment of the present invention to a subject. In variations, the maximum dosage of the formulation is split at least once into doses and the formulation is delivered in proportion to the number of times the formulation is split.

For example, if the dose is split once, the subject is administered a half of the maximum dose at a first time. The second dose is administered to the subject within 12 hours, preferably within 6 hours, more preferably within 3 hours, and most preferably within an hour, of the first dose. Most preferred is to administer the split dose as part of a combination dosage form, either as part of the same form or two separate forms, within 5 minutes, or at the same time.

Exemplary embodiments of the present invention have now been described in accordance with the above advantages. It will be appreciated that these examples are merely illustrative of the invention. Many variations and modifications will be apparent to those skilled in the art. Moreover, any of the embodiments illustrated above and below stand independently or features may be combined to achieve preferred embodiments.

The exemplary formulations are presented to further describe the present invention.

### EXAMPLE 1

### Core Formulation

| | Amount per |
|---|---|
| Ingredient | Day (mg) |
| Thiamin | 100 |
| Niacin | 250 |
| Pantothenic Acid | 250 |
| Huperzine | 0.15 |
| DMAE bitartrate | 300 |

### EXAMPLE 2

### Core Formulation, Additional Nutraceuticals, and Excipients

| | Amount per |
|---|---|
| Ingredient | Day (mg) |
| Thiamin | 100 |
| Niacin | 250 |
| Pantothenic Acid | 250 |
| Huperzine | 0.15 |
| DMAE bitartrate | 300 |
| Folic Acid | 2 |
| Pyridoxine | 20 |
| Vitamin B12 | 1 |
| Magnesium Stearate | 12 |
| Silica | 25 |
| Rice Powder | 400 |

### EXAMPLE 3

### Core Formulation, Additional Nutraceuticals, and Excipients

| | Amount per |
|---|---|
| Ingredient | Day (mg) |
| Thiamin | 100 |
| Niacin | 250 |
| Pantothenic Acid | 250 |
| Huperzine | 0.15 |
| DMAE bitartrate | 300 |
| Vinpocetine | 20 |
| Magnesium Stearate | 12 |
| Silica | 25 |
| Rice Powder | 400 |

### EXAMPLE 4

### Additional Nutraceuticals and Excipients

| | Amount per |
|---|---|
| Ingredient | Day (mg) |
| Thiamin | 100 |
| Niacin | 250 |
| Pantothenic Acid | 250 |
| Huperzine | 0.15 |
| DMAE bitartrate | 300 |
| Vinpocetine | 20 |
| Acetyl-L-Carnitine HCL | 1000 |
| Magnesium Stearate | 12 |
| Silica | 25 |
| Rice Powder | 400 |

### EXAMPLE 5

### Core Formulation, Additional Nutraceuticals, and Excipients

| | Amount per |
|---|---|
| Ingredient | Day (mg) |
| Thiamin | 100 |
| Niacin | 250 |
| Pantothenic Acid | 250 |
| Huperzine | 0.15 |
| DMAE bitartrate | 300 |
| Vinpocetine | 20 |
| L-Pyroglutamic Acid | 500 |
| Magnesium Stearate | 12 |
| Silica | 25 |
| Rice Powder | 400 |

### EXAMPLE 6

### Core Formulation, Additional Nutraceuticals, and Excipients

| | Amount per |
|---|---|
| Ingredient | Day (mg) |
| Thiamin | 100 |
| Niacin | 250 |
| Pantothenic Acid | 250 |
| Huperzine | 0.15 |
| DMAE bitartrate | 300 |
| Vinpocetine | 20 |
| Acetyl-L-Carnitine HCL | 1000 |
| L-Pyroglutamic Acid | 500 |
| Magnesium Stearate | 12 |
| Silica | 25 |
| Rice Powder | 400 |

### EXAMPLE 7

### Core Formulation, Additional Nutraceuticals, and Excipients

| | Amount per |
|---|---|
| Ingredient | Day (mg) |
| Thiamin | 100 |
| Niacin | 250 |
| Pantothenic Acid | 250 |
| Huperzine | 0.15 |
| DMAE bitartrate | 300 |
| Rhodiola | 300 |
| Magnesium Stearate | 12 |
| Silica | 25 |
| Rice Powder | 400 |

### EXAMPLE 8

### Core Formulation, Additional Nutraceuticals, and Excipients

| | Amount per |
|---|---|
| Ingredient | Day (mg) |
| Thiamin | 100 |
| Niacin | 250 |
| Pantothenic Acid | 250 |
| Huperzine | 0.15 |
| DMAE bitartrate | 300 |
| Pyridoxine | 20 |
| Folic Acid | 2 |
| Vitamin B₁₂ | 1 |
| PS | 50 |
| Magnesium Stearate | 12 |
| Silica | 25 |
| Rice Powder | 400 |

### EXAMPLE 9

### Core Formulation, Additional Nutraceuticals, and Excipients

| | Amount per |
|---|---|
| Ingredient | Day (mg) |
| Thiamin | 100 |
| Niacin | 250 |
| Pantothenic Acid | 250 |
| Huperzine | 0.15 |
| DMAE bitartrate | 300 |
| L-Theanine | 100 |
| Magnesium Stearate | 12 |
| Silica | 25 |
| Rice Powder | 400 |

### EXAMPLE 10

### Core Formulation, Additional Nutraceuticals, and Excipients

| | Amount per |
|---|---|
| Ingredient | Day (mg) |
| Thiamin | 100 |
| Niacin | 250 |
| Pantothenic Acid | 250 |
| Huperzine | 0.15 |
| DMAE bitartrate | 300 |
| PS | 50 |
| Folic Acid | 2 |
| Vitamin B12 | 1 |
| Vitamin B6 | 20 |
| Magnesium Stearate | 21 |
| Silica | 50 |
| Dextrose | 170 |
| Stearic Acid | 70 |
| Microcrystalline Cellulose | 240 |
| Croscarmellose Sodium | 62 |

### EXAMPLE 11

### Core Formulation, Additional Nutraceuticals, and Excipients

| | Amount per |
|---|---|
| Ingredient | Day (mg) |
| Thiamin | 100 |
| Niacin | 250 |
| Pantothenic Acid | 250 |
| Huperzine | 0.15 |
| DMAE bitartrate | 300 |
| L-Theanine | 100 |
| Magnesium Stearate | 18 |
| Silica | 36 |
| Dextrose | 100 |
| Stearic Acid | 70 |
| Microcrystalline Cellulose | 236 |
| Croscarmellose Sodium | 80 |

### EXAMPLE 12

### Core Formulation, Additional Nutraceuticals, and Excipients

| | Amount per |
|---|---|
| Ingredient | Day (mg) |
| Thiamin | 100 |
| Niacin | 250 |
| Pantothenic Acid | 250 |
| Huperzine | 0.15 |
| DMAE bitartrate | 300 |
| Rhodiola Rosea Extract | 300 |
| Magnesium Stearate | 24 |
| Silica | 50 |
| Microcrystalline Cellulose | 250 |
| Stearic Acid | 76 |
| Dicalcium phosphate | 140 |
| Croscarmellose Sodium | 80 |

Another example of the beneficial and synergistic effects of the core ingredients is provided in a controlled clinical trial (herein known as “the Study”) sponsored by Natrol, Inc. of Chatsworth, Calif., USA. The study assessed the cognitive benefits of several nutraceutical compositions having the core ingredients, and in particular, whether the combination of core ingredients and/or additional nutraceuticals has a direct or indirect affect on neural functions and/or can improve cognitive performance in normal adults. The study was an IRB approved, double blind, placebo-controlled protocol over a six-week trial period. The sample size of the study was greater or equal to fifty (N≧50) per group and over 400 subjects completed the study. There were five treatment groups (“test group”) and one control group (“placebo group”) in this study. The age range of the subjects was from 20 years of age to 70 years of age.

Testing of cognitive function was conducted at week zero (baseline) and weekly for the six weeks thereafter in which the test group was administered test compositions. Cognitive function was measured using clinically validated, internet-based cognitive assessment tests selected from the NCAT (Neuro-cognitive Chronometric Assessment Technology) test battery, also known as the Cognometer, developed by Cognitive Labs, Inc. (Mountain View, Calif.). In particular, the study employed Cognometer Test 4 to assess executive function and Cognometer Test 10 to assess immediate memory. Each test type is further discussed below.

The executive decision test is a complex choice reaction time task assessing executive cognitive function including sustained attention and ability to correctly make rapid decisions. The test involves presenting a word and picture simultaneously and requires a decision to state if they are the same or different. A reversal cue is randomly presented that requires the person to respond opposite of the correct response. This test requires suppression or inhibition of a learned response and then a reversal (known as “task shifting”) of the response contingency creating a high order of cognitive load. As should be understood to one skilled in the art, task shifting is generally equated to mental flexibility. The speed of switching from one task or one response mode to another is equated with mental quickness and flexibility and decision-making abilities.

The test for immediate memory is a memory item scanning and an immediate recall task. Generally, the test group is provided with a string of stimulus target items (e.g., letters) to be remembered and then followed up with an inquiry or probe item. The test group must decide whether the probe item was a member of the previous target list. Reaction time, or speed of scanning and recall (i.e., memory processing) in this test is considered to be a reliable indicator of verbal and visuo-spatial memory.

Study results suggest a nutrient effect on cognitive function. Specifically, the study found a synergistic effect with the core formulation. With regard to the executive function test, the core formulation showed improvements at study completion compared to baseline. This finding suggests improved mental quickness and flexibility, improved decision-making, improved decision-making speed, improved cognitive processing, improved decision-making speed in a demanding cognitive task, improved attention or focus, and improved attention or focus on a demanding cognitive task.

What is claimed:

1. A nutraceutical composition suitable to support cognitive function comprising:
huperzine A, or a nutraceutically acceptable form thereof;
greater than 38 mg of DMAE, or a nutraceutically acceptable form thereof that provides greater than 38 mg of DMAE;
thiamin, or a nutraceutically acceptable form thereof;
niacin, or a nutraceutically acceptable form thereof;
pantothenic acid, or a nutraceutically acceptable form thereof; and
optionally one or more nutraceutically acceptable excipients.

2. The composition according to claim 1, wherein the composition comprises:
about 0.01 mg to about 1.0 mg of huperzine A, or the nutraceutically acceptable form thereof;
greater than 38 mg to about 500 mg of DMAE, or the nutraceutically acceptable form thereof, wherein if the nutraceutically acceptable form is DMAE bitartrate, the amount is greater than 100 mg to about 1500 mg;
about 25 mg to about 125 mg of thiamin, or the nutraceutically acceptable form thereof;
about 100 mg to about 300 mg of niacin, or the nutraceutically acceptable form thereof; and
greater than 100 mg to about 300 mg of pantothenic acid, or the nutraceutically acceptable form thereof.

3. The composition according to claim 2, wherein the composition comprises:
about 0.15 mg of huperzine A, or the nutraceutically acceptable form thereof;
about 111 mg of DMAE;
about 100 mg of thiamin, or the nutraceutically acceptable form thereof;
about 250 mg of niacin, or the nutraceutically acceptable form thereof; and
about 250 mg of pantothenic acid, or the nutraceutically acceptable form thereof.

4. The composition according to claim 2, wherein the composition consists essentially of:
about 0.15 mg of huperzine A, or the nutraceutically acceptable form thereof;
about 111 mg of DMAE;
about 100 mg of thiamin, or the nutraceutically acceptable form thereof;
about 250 mg of niacin, or the nutraceutically acceptable form thereof; and
about 250 mg of pantothenic acid, or the nutraceutically acceptable form thereof.

5. The composition according to claim 1, further comprising:
an amount of vinpocetine, or a nutraceutically acceptable form thereof.

6. The composition according to claim 5, further comprising:
an amount of L-pyroglutamic acid, or a nutraceutically acceptable form thereof.

7. The composition according to claim 4, further comprising:
an amount of acetyl-L-carnitine or a nutraceutically acceptable form thereof.

8. The composition according to claim 5, further comprising:
an amount of acetyl-L-carnitine or a nutraceutically acceptable form thereof.

9. The composition according to claim 8, further comprising:
an amount of L-pyroglutamic acid, or a nutraceutically acceptable form thereof.

10. The composition according to claim 1, further comprising:
an amount of any two selected from the group consisting of vinpocetine or a nutraceutically acceptable form thereof, acetyl-L-carnitine or a nutraceutically acceptable form thereof, and L-pyroglutamic acid or a nutraceutically acceptable form thereof.

11. The composition according to claim 1, further comprising:
an amount of vinpocetine or a nutraceutically acceptable form thereof;
an amount of acetyl-L-carnitine or a nutraceutically acceptable form thereof; and
an amount of L-pyroglutamic acid or a nutraceutically acceptable form thereof.

12. The composition according to claim 2, further comprising:
about 10 to about 30 mg of vinpocetine or a nutraceutically acceptable form thereof;
about 400 to about 1600 mg of acetyl-L-carnitine or the nutraceutically acceptable form thereof; and
about 250 to about 750 mg of L-pyroglutamic acid or the nutraceutically acceptable form thereof.

13. The composition according to claim 3, further comprising:
about 20 mg of vinpocetine or a nutraceutically acceptable form thereof;
about 800 mg of acetyl-L-carnitine or a nutraceutically acceptable form thereof; and
about 500 mg of L-pyroglutamic acid or a nutraceutically acceptable form thereof.

14. The composition according to claim 3, wherein the composition consists essentially of:
the about 0.15 mg of huperzine A, or the nutraceutically acceptable form thereof;
the about 111 mg of DMAE;
the about 100 mg of thiamin, or the nutraceutically acceptable form thereof;
the about 250 mg of niacin, or the nutraceutically acceptable form thereof,
the about 250 mg of pantothenic acid, or the nutraceutically acceptable form thereof;
about 20 mg of vinpocetine or a nutraceutically acceptable form thereof;
about 800 mg of acetyl-L-carnitine or a nutraceutically acceptable form thereof; and
about 500 mg of L-pyroglutamic acid or a nutraceutically acceptable form thereof.

15. The composition according to claim 1, further comprising:
an amount of rhodiola rosea root or a nutraceutically acceptable form thereof.

16. The composition according to claim 2, further comprising:
about 200 to about 600 mg of rhodiola rosea root extract or a nutraceutically acceptable form thereof.

17. The composition according to claim 3, further comprising:
about 300 mg of rhodiola rosea root extract or a nutraceutically acceptable form thereof.

18. The composition according to claim 3, wherein the composition consists essentially of:
the about 0.15 mg of huperzine A, or the nutraceutically acceptable form thereof;
the about 111 mg of DMAE;
the about 100 mg of thiamin, or the nutraceutically acceptable form thereof;
the about 250 mg of niacin, or the nutraceutically acceptable form thereof;
the about 250 mg of pantothenic acid, or the nutraceutically acceptable form thereof; and
about 300 mg of rhodiola rosea root extract or a nutraceutically acceptable form thereof.

19. The composition according to claim 2, further comprising:
about 50 to about 250 mg of L-theanine, or a nutraceutically acceptable form thereof.

20. The composition according to claim 3, further comprising:
about 100 mg of L-theanine, or a nutraceutically acceptable form thereof.

21. The composition according to claim 3, wherein the composition consists essentially of:
the about 0.15 mg of huperzine A, or the nutraceutically acceptable form thereof;
the about 111 mg of DMAE;
the about 100 mg of thiamin, or the nutraceutically acceptable form thereof;
the about 250 mg of niacin, or the nutraceutically acceptable form thereof;
the about 250 mg of pantothenic acid, or the nutraceutically acceptable form thereof;
about 100 mg of L-theanine, or a nutraceutically acceptable form thereof.

22. The composition according to claim 1, further comprising:
an amount of pyridoxine, or a nutraceutically acceptable form thereof;
an amount of folic acid or a nutraceutically acceptable form thereof;
an amount of vitamin B₁₂ or a nutraceutically acceptable form thereof; and
an amount of phosphatidylserine, or a nutraceutically acceptable form thereof.

23. The composition according to claim 2, further comprising:
about 10 to about 50 mg of pyridoxine, or a nutraceutically acceptable form thereof;
about 0.4 to about 2.0 mg of folic acid or a nutraceutically acceptable form thereof;
about 0.5 to about 1.5 mg of vitamin B₁₂ or a nutraceutically acceptable form thereof; and
about 50 to about 300 mg of phosphatidylserine, or the nutraceutically acceptable form thereof.

24. The composition according to claim 3, wherein the composition consists essentially of:
the about 0.15 mg of huperzine A, or the nutraceutically acceptable form thereof;
the about 111 mg of DMAE;
the about 100 mg of thiamin, or the nutraceutically acceptable form thereof;
the about 250 mg of niacin, or the nutraceutically acceptable form thereof;
the about 250 mg of pantothenic acid, or the nutraceutically acceptable form thereof;
about 20 mg of pyridoxine, or a nutraceutically acceptable form thereof;
about 2 mg of folic acid or a nutraceutically acceptable form thereof;
about 1 mg of vitamin B₁₂ or a nutraceutically acceptable form thereof; and
about 50 mg of phosphatidylserine, or a nutraceutically acceptable form thereof.

25. The composition according to claim 1, further comprising:
an amount of L-theanine, or a nutraceutically acceptable form thereof.

26. The composition according to claim 2, further comprising:
about 50 to about 250 mg of L-theanine, or a nutraceutically acceptable form thereof.

27. The composition according to claim 3, further comprising:
about 100 mg of L-theanine, or a nutraceutically acceptable form thereof.

28. The composition according to claim 3, wherein the composition consists essentially of the about 0.15 mg of huperzine A, or nutraceutically acceptable form thereof, the about 111 mg of DMAE, the about 100 mg of thiamin, or nutraceutically acceptable form thereof, the about 250 mg of niacin, or nutraceutically acceptable form thereof, and the about 250 mg of pantothenic acid, or nutraceutically acceptable form thereof, and the about 100 mg of L-theanine, or nutraceutically acceptable form thereof.

29. The composition according to claim 1, wherein the composition is in a form for administration orally, dermally, or parenterally.

30. The composition according to claim 1, wherein the composition is packaged in one member of the group consisting of a tablet, a capsule, a powder, a chewing gum, a bar, and a liquid.

31. The composition according to claim 1, wherein DMAE and huperzine are blended prior to their addition to the composition.

32. The composition according to claim 1, wherein the tablet is coated.

33. The composition according to claim 7, wherein the acetyl-L-carnitine is in the form of a nutraceutically acceptable form selected from the group consisting of acetyl-L-carnitine hydrochloride, acetyl-L-carnitine hydrogen fumarate, acetyl-L-carnitine taurinate hydrochloride, L-ornithine acetyl-L-carnitine dihydrochloride, acetyl-L-carnitine arginate dihydrochloride and L-leucine acetyl-L-carnitine hydrochloride.

34. The composition according to claim 1, wherein the nutraceutically acceptable form of niacin is nicotinic acid.

35. The composition according to claim 34, wherein the composition comprises:
about 0.01 mg to about 1.0 mg of huperzine A, or the nutraceutically acceptable form thereof;
greater than 38 mg to about 500 mg of DMAE, or the nutraceutically acceptable form thereof, wherein if the nutraceutically acceptable form is DMAE bitartrate, the amount is greater than 100 mg to about 1500 mg;
about 25 mg to about 125 mg of thiamin, or the nutraceutically acceptable form thereof;
no greater than 35 mg of nicotinic acid; and
greater than 100 mg to about 300 mg of pantothenic acid, or the nutraceutically acceptable form thereof.

36. The composition according to claim 35, wherein the composition comprises:
about 0.15 mg of huperzine A, or the nutraceutically acceptable form thereof;
about 111 mg of DMAE, or the nutraceutically acceptable form thereof;
about 100 mg of thiamin, or the nutraceutically acceptable form thereof;
about 25 mg of nicotinic acid; and
about 250 mg of pantothenic acid, or the nutraceutically acceptable form thereof.

37. The composition according to claim 36, wherein the composition consists essentially of:
the about 0.15 mg of huperzine A, or the nutraceutically acceptable form thereof;
the about 111 mg of DMAE, or the nutraceutically acceptable form thereof;
the about 100 mg of thiamin, or the nutraceutically acceptable form thereof;
the about 25 mg of nicotinic acid; and
the about 250 mg of pantothenic acid, or the nutraceutically acceptable form thereof.

38. A method for improvement of cognitive function comprising:
administering a composition comprising an amount of huperzine A, or a nutraceutically acceptable form thereof, greater than 38 mg of DMAE, or a nutraceutically acceptable form thereof that provides greater than 38 mg of DMAE, an amount of thiamin, or a nutraceutically acceptable form thereof, an amount of niacin, or a nutraceutically acceptable form thereof, an amount of pantothenic acid, or a nutraceutically acceptable form thereof, and optionally an amount of one or more nutraceutically acceptable excipients.

39. The method according to claim 38, wherein the administering is in the form of at least one tablet or capsule taken orally.

40. The method according to claim 39, wherein the at least one tablet or capsule is one, two, three, four, five, or six doses taken daily.

41. The method according to claim 38, wherein the administering achieves at least one result selected from the group consisting of improved learning, supported learning, improved thinking, supported thinking, improved judging, supported judging, promoted focus, promoted attention, promoted alertness, promoted awareness, and promoted mental clarity.

42. The method according to claim 38, wherein the composition consists essentially of:
about 0.01 mg to about 1.0 mg of huperzine A, or nutraceutically acceptable form thereof;
greater than 38 mg to about 500 mg of DMAE, or nutraceutically acceptable form thereof that provides greater than 38 mg of DMAE;
about 25 mg to about 125 mg of thiamin, or nutraceutically acceptable form thereof;
about 100 mg to about 300 mg of niacin, or nutraceutically acceptable form thereof; and
greater than 100 mg to about 300 mg of pantothenic acid, or nutraceutically acceptable form thereof.

43. The method according to claim 38, wherein the method further comprises administering an amount of vinpocetine or a nutraceutically acceptable form thereof, acetyl-L-carnitine or a nutraceutically acceptable form thereof, L-pyroglutamic acid or a nutraceutically acceptable form thereof, an amount of pyridoxine, or a nutraceutically acceptable form thereof; folic acid or a nutraceutically acceptable form thereof, vitamin B₁₂ or a nutraceutically acceptable form thereof; and phosphatidylserine, or a nutraceutically acceptable form thereof.

44. The method according to claim 43, wherein said composition consists essentially of about 0.15 mg of huperzine A, or nutraceutically acceptable form thereof; about 111 mg of DMAE, or nutraceutically acceptable form thereof; about 100 mg of thiamin, or nutraceutically acceptable form thereof; about 250 mg of niacin, or nutraceutically acceptable form thereof, about 250 mg of pantothenic acid, or nutraceutically acceptable form thereof; about 20 mg of vinpocetine or nutraceutically acceptable form thereof; about 800 mg of acetyl-L-carnitine or nutraceutically acceptable form thereof; and about 500 mg of L-pyroglutamic acid or nutraceutically acceptable form thereof.

45. A method for improving cognitive function, comprising administrating to a human the composition of claim 1.
